(19) United States
(12) Patent Application Publication
(10) Pub. No.: US 2023/0165520 A1
(43) Pub. Date: Jun. 1, 2023

(54) METHODS AND SYSTEMS FOR PREDICTING THE EFFECT OF INHALED AND INFUSED ANESTHETICS

(71) Applicants: BioVentures, LLC, Litte Rock, AR (US); Arkansas Children's Hospital Research Institute, Little Rock, AR (US); Board of Trustees of the University of Arkansas, Little Rock, AR (US)

(72) Inventors: KEVIN SEXTON, Little Rock, AR (US); JINGXIAN WU, Little Rock, AR (US); MORTEN JENSEN, Little Rock, AR (US); HANNA JENSEN, Little Rock, AR (US); MELVIN DASSINGER, III, Little Rock, AR (US); KAYLEE HENRY, Little Rock, AR (US); JOSEPH SANFORD, Little Rock, AR (US); ALI AL-ALAWI, Little Rock, AR (US); PATRICK BONASSO, Little Rock, AR (US)

(21) Appl. No.: 17/919,474

(22) PCT Filed: Apr. 13, 2021
(86) PCT No.: PCT/US2021/026977

§ 371 Date: Oct. 17, 2022

Related U.S. Application Data
(60) Provisional application No. 63/011,654, filed on Apr. 17, 2020.

(51) Int. Cl. A61B 5/00; A61P 23/00

(52) U.S. Cl. CPC A61B 5/4821; A61P 23/00; A61B 5/318

(57) ABSTRACT

Disclosed herein are systems and methods for non-invasively predicting a hemodynamic state and/or an anesthetic depth of a patient, such as a pediatric patient. The method may include receiving a peripheral venous pressure (PVP) waveform from the patient, cleaning the PVP waveform, transforming the PVP waveform into the frequency domain, and automatically predicting the hemodynamic state and/or the anesthetic depth of the patient.

### CROSS-REFERENCE TO RELATED APPLICATIONS

This application claims priority to U.S. Provisional Application No. 63/011,654, filed Apr. 17, 2020, the contents of which are entirely incorporated by reference herein.

### GOVERNMENT INTEREST STATEMENT

This invention was made with government support under 1U54TR001629-01A1 awarded by the National Institutes of Health and ECCS1711087 awarded by the National Science Foundation. The government has certain rights in the invention.

### FIELD

The present disclosure relates to systems and methods for predicting the effect of inhaled and infused anesthetics on a patient. More specifically, the disclosure relates to predicting the effect of inhaled and infused anesthetics using peripheral venous pressure waveforms.

### BACKGROUND

The depth of a patient's anesthesia in the hemorrhagic portion of the surgery is controlled by altering the minimum alveolar concentration (MAC) of an inhaled anesthetic, where a higher MAC corresponds to a higher dosage of the anesthetic. The depth in the non-hemorrhagic portion of the surgery is controlled by applying bolus dosages of an infused anesthetic. Anesthetic drugs that patients receive before any intervention change the physiology of the blood circulation in the vessels causing vasodilation to the vessels.

Previous forms of anesthesia depth assessors have been developed for adult patients, but they are not minimally invasive and therefore not appropriate for pediatric patients. Traditional clinical signs such as hypertension, tachycardia and lacrimation are unreliable indicators of depth of anesthesia. Early techniques based on real time signal processing such as the raw or summated EEG, and lower oesophageal contractility, were unreliable. Many methods use a dimensionless monotonic index as a measure of anesthetic depth.

Therefore, there is a need for a minimally invasive method of predicting the effect of inhaled and infused anesthetics, particularly for the pediatric population.

### SUMMARY

This disclosure provides a method of predicting the effect of inhaled and infused anesthetics using PVP waveforms.

In an aspect, a method of predicting a hemodynamic state of a patient being administered an anesthetic may include receiving a peripheral venous pressure (PVP) waveform from the patient, cleaning the PVP waveform, transforming the PVP waveform into the frequency domain, and automatically predicting a hemodynamic state of the patient. The prediction may be made using a k-nearest neighbor (k-NN), neural network, random forest, SVM, naïve Bayes, and/or K-means model. The method may further include acquiring the PVP waveform using a peripheral intravenous catheter linked to a pressure transducer and/or measuring the patient's electrocardiography (ECG) waveform.

Cleaning the PVP waveform may include sectioning the PVP waveform at a pre-selected length of time to create one or more segments, calculating a remainder of the PVP waveform divided by the pre-selected length of time, removing any last points of the PVP waveform that are equal to the PVP waveform remainder, calculating the mean and the standard deviation for each segment, and removing a segment if there is at least one point outside a set number of standard deviations selected by the user.

The hemodynamic state may be a hypervolemic state, an euvolemic state or a hypovolemic state. The anesthetic may be an infused anesthetic, such as propofol, etomidate, benzodiazepines, fentanyl, rem ifentanil, sufentanyl, morphine, hydromorphone, phenobarbital, pentobarbital, methohexital, ketamine, esketamine, precedex, lidocaine, bupivacaine, ropivacaine, tetracaine, chloroprocaine, clonidine, fentanyl, hydromorphone, morphine, epinephrine, sodium bicarbonate, or glucocorticoids. The patient may be a pediatric patient.

In another aspect, a method of predicting an anesthetic depth of a patient being administered an anesthetic may include receiving a peripheral venous pressure (PVP) waveform from the patient, cleaning the PVP waveform, transforming the PVP waveform into the frequency domain, and automatically predicting the anesthetic depth of the patient. The automatic prediction may be made using a k-nearest neighbor (k-NN), neural network, random forest, SVM, naïve Bayes, and/or K-means model. The method may further include acquiring the PVP waveform using a peripheral intravenous catheter linked to a pressure transducer. The method may also include measuring the patient's ECG and/or determining ECG and PVP waveform coefficients at the heart rate and respiratory rate frequencies.

Cleaning the PVP waveform may include sectioning the PVP waveform at a pre-selected length of time to create one or more segments, calculating a remainder of the PVP waveform divided by the pre-selected length of time, removing any last points of the PVP waveform that are equal to the PVP waveform remainder, calculating the mean and the standard deviation for each segment, and removing a segment if there is at least one point outside a set number of standard deviations selected by the user.

The anesthetic depth may be a minimum alveolar concentration (MAC) dosage. The anesthetic may be an inhaled anesthetic such as isoflurane, sevoflourane, desflurane, halothane, or nitrous oxide. The patient may be a pediatric patient.

Another aspect provided herein is a device having at least one non-transitory computer readable medium storing instructions which when executed by at least one processor, cause the at least one processor to: receive a peripheral venous pressure (PVP) waveform from a patient administered an anesthetic, clean the PVP waveform, transform the PVP waveform into the frequency domain, and automatically predict a hemodynamic state of the patient and/or an anesthetic depth of the patient. The automatic prediction may be made using a k-nearest neighbor (k-NN), neural network, random forest, SVM, naïve Bayes, and/or K-means model. The patient may be a pediatric patient. The hemodynamic state of the patient and/or the anesthetic depth of the patient may be predicted automatically. The device may further include a peripheral intravenous catheter linked to a pressure transducer to acquire the PVP waveform. The hemodynamic state may be a hypervolemic state, an euvolemic state or a hypovolemic state and the anesthetic depth may be a minimum alveolar concentration (MAC) dosage. The anesthetic may be an infused anesthetic such as propofol, etomidate, benzodiazepines, fentanyl, rem ifentanil, sufentanyl, morphine, hydromorphone, phenobarbital, pentobarbital, methohexital, ketamine, esketamine, precedex, lidocaine, bupivacaine, ropivacaine, tetracaine, chloroprocaine, clonidine, fentanyl, hydromorphone, morphine, epinephrine, sodium bicarbonate, or glucocorticoids or an inhaled anesthetic such as isoflurane, sevoflourane, desflurane, halothane, or nitrous oxide.

### BRIEF DESCRIPTION OF THE DRAWINGS

The description will be more fully understood with reference to the following figures and data graphs, which are presented as various embodiments of the disclosure and should not be construed as a complete recitation of the scope of the disclosure. It is noted that, for purposes of illustrative clarity, certain elements in various drawings may not be drawn to scale. Understanding that these drawings depict only exemplary embodiments of the disclosure and are not therefore to be considered to be limiting of its scope, the principles herein are described and explained with additional specificity and detail through the use of the accompanying drawings in which:

FIG. 1 is a diagram of the prediction method in one example.

FIG. 2A shows an example euvolemic patient's preoperative peripheral venous pressure (PVP) waveform in time domain.

FIG. 2B shows an example euvolemic patient's intraoperative PVP waveform.

FIG. 2C shows an example euvolemic patient's preoperative frequency domain PVP and piezoelectric waveforms.

FIG. 2D shows an example euvolemic patient's intraoperative frequency domain PVP and piezoelectric waveforms.

FIG. 3A shows an example isoflurane patient's PVP waveform in the time domain for MAC group 1.

FIG. 3A shows an example isoflurane patient's PVP waveform in the time domain for MAC group 2.

FIG. 3A shows an example isoflurane patient's PVP waveform in the frequency domain and EKG waveform for MAC group 1.

FIG. 3A shows an example isoflurane patient's PVP waveform in the frequency domain and EKG waveform for MAC group 2.

FIG. 4 illustrates example system embodiments.

FIG. 5 illustrates an example machine learning environment.

FIG. 6 is an example of movement interfering with collection of the PVP waveform.

FIG. 7 is an example of cleaning the PVP waveform, where the box with the cross encloses an unwanted data section that will be removed.

FIG. 8A is a receiver operating characteristic (ROC) curve plotted as 1-specificity vs sensitivity for propofol.

FIG. 8B is a ROC curve plotted as 1-specificity vs sensitivity for MAC classification.

Reference characters indicate corresponding elements among the views of the drawings. The headings used in the figures do not limit the scope of the claims.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure. Thus, the following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be references to the same embodiment or any embodiment; and, such references mean at least one of the embodiments.

Reference to “one embodiment”, “an embodiment”, or “an aspect” means that a particular feature, structure, or characteristic described in connection with the embodiment is included in at least one embodiment of the disclosure. The appearances of the phrase “in one embodiment” or “in one aspect” in various places in the specification are not necessarily all referring to the same embodiment, nor are separate or alternative embodiments mutually exclusive of other embodiments. Moreover, various features are described which may be exhibited by some embodiments and not by others.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any example term. Likewise, the disclosure is not limited to various embodiments given in this specification.

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims, or can be learned by the practice of the principles set forth herein.

Provided herein are methods of predicting the effect of anesthetics on a patient using peripheral venous pressure (PVP) waveforms. The methods of predicting the effect of anesthetics on a patient may be used to prevent overdosage or underdosage of anesthesia during a pediatric medical operation. In some examples, infused and inhaled anesthetics may have an impact on the PVP waveforms and machine learning may be used to automatically identify how anesthetics are affecting a patient by analyzing the patient's PVP waveforms. The method may be nearly instantaneous, minimally invasive, work with both infused and inhaled anesthetics, and be applicable to pediatric populations.

Analysis of peripheral venous pressure (PVP) waveforms is a novel method of monitoring intravascular volume, especially in cases of dehydration and hemorrhage. PVP has been shown to be a predictor of dehydration in pediatric patients. However, PVP waveforms can potentially be confounded by parameters other than volume status, such as anesthetic agents, while collecting the data. Anesthetic drugs, inhaled or infused, influence the PVP signal significantly.

The methods provided herein determined a significant relationship between both infused and inhaled anesthetics and the PVP waveform, as the PVP signal is influenced by the different hemodynamics states of the body.

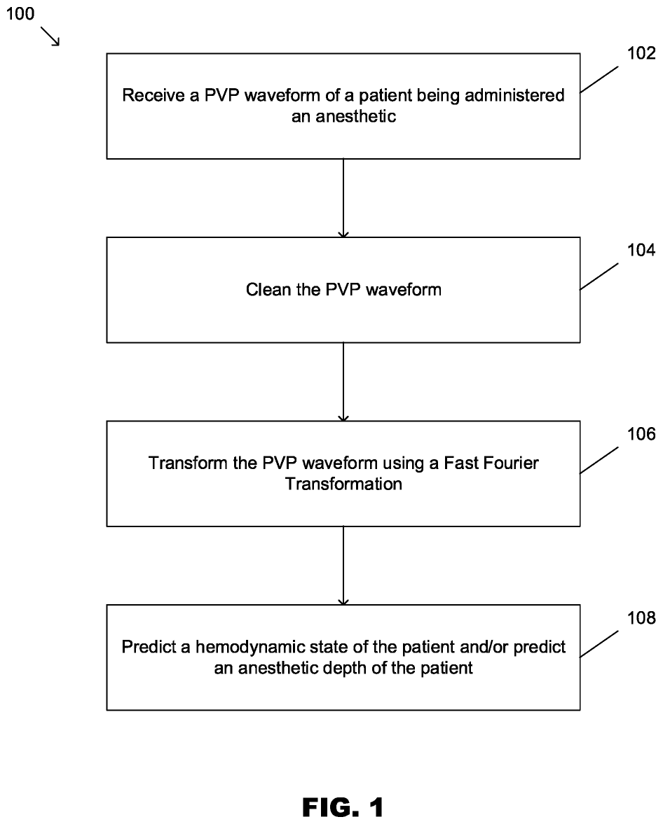

The overall framework of the prediction method 100 is shown in FIG. 1. At step 102, the prediction method 100 may include receiving a peripheral venous pressure (PVP) waveform from a patient being administered an anesthetic. In at least one example, the patient is a pediatric patient. In additional examples, the pediatric patient may be an infant. The anesthetic may be an infused anesthetic or an inhaled anesthetic. Non-limiting examples of inhaled anesthetics include isoflurane, sevoflurane, desflurane, halothane, and nitrous oxide. Isoflurane causes vasodilation in the peripheral blood vessels and alters the blood flow. The infused anesthetic may be an infused gamma-aminobutyric acid (GABA) agonist anesthetic, an infused narcotic, an infused barbiturate, an infused NMDA antagonist, an infused alpha agonist, or an infused neuraxial anesthetic. Non-limiting examples of GABA agonists include propofol, etomidate, and benzodiazepines. Propofol is an anesthetic drug that causes immediate vasodilation and relaxes the patient's vessels, which decreases the pressure in the vessels. Non-limiting examples of infused narcotics include fentanyl, rem ifentanil, sufentanyl, morphine, and hydromorphone. Non-limiting examples of infused barbiturates include phenobarbital, pentobarbital, and methohexital. Non-limiting examples of infused NMDA antagonists include ketamine and esketamine. Non-limiting examples of infused alpha agonists include precedex. Non-limiting examples of neuraxial anesthetics include lidocaine, bupivacaine, ropivacaine, tetracaine, chloroprocaine, clonidine, fentanyl, hydromorphone, morphine, epinephrine, sodium bicarbonate, and glucocorticoids.

In various examples, a device may include an apparatus for acquiring the PVP waveform and at least one processor for performing the steps of the method 100. The device may continuously measure the PVP waveform and predict the anesthetic depth in the patient before and during a medical operation. In some examples, the PVP waveform may be acquired using a peripheral intravenous catheter linked to a pressure transducer. PVP can be measured via a peripheral IV, making it easy to access and measure compared to central venous pressure (CVP). In at least some examples, the PVP waveform may be measured via a peripheral IV in the arms or legs of the patient or at any location on the patient that may receive a peripheral IV. CVP is traditionally used in assessing the overall circulatory status of a patient in an intensive care or operative setting, and to guide resuscitation. Several studies have shown that CVP and PVP correlate significantly. However, use of PVP waveforms is a less invasive method of measuring volume status. The PVP waveform may be acquired by any method known in the art. In some examples, the PVP waveform may be acquired through a piezoelectric crystal. In additional examples, the PVP waveform may be acquired transcutaneously.

At step 104, the method 100 may include cleaning the PVP waveform. Cleaning the PVP waveform may remove unwanted motion artifacts. In various examples, the PVP waveform may be cleaned automatically. Cleaning the PVP waveform automatically may include sectioning the PVP waveform at a pre-selected length of time to create one or more segments, calculating a remainder of the PVP waveform divided by the pre-selected length of time, removing any last points of the PVP waveform that are equal to the PVP waveform remainder, calculating the mean and the standard deviation for each segment, and removing a segment if there is at least one point outside a set number of standard deviations selected by the user.

At step 106, the method 100 may include transforming the PVP waveform into the frequency domain. In some examples, the PVP waveform may be transformed using a Fast Fourier Transformation (FFT). The venous system is highly compliant and can accommodate large changes in volume with minimal changes in pressure. However, the detection of the subtle changes in PVP waveforms as a result of volume loss is made possible due to signal amplifying technologies that can extract hemodynamic signals in the frequency domain by using FFT. The frequency domain PVP signals may then be analyzed with advanced statistical and machine learning algorithms. Venous waves are generated by the cardiac cycle and propagated as harmonics. The f1 waveform which correlates with the heart rate, has been shown to be affected already by very mild hypovolemia. The FFT of a PVP waveform correlates with volume status more sensitively than standard vital signs monitoring. However, despite the robust evidence of the correlation between PVP waveforms and volume status, both the exact mechanism behind this link, and potential confounding parameters have not been thoroughly investigated.

At step 108, the method 100 may include automatically predicting a hemodynamic state of the patient and/or automatically predicting an anesthetic depth of the patient. In some examples, the method may automatically predict a hemodynamic state and/or automatically predict an anesthetic depth using a k-nearest neighbor (k-NN), neural network, random forest, SVM, naïve Bayes, and/or K-means model. In some examples, the prediction of the hemodynamic state or the anesthetic depth may prevent overdosage or underdosage of anesthesia during a medical operation, in particular a pediatric medical operation. Predicting the hemodynamic state of the patient or predicting the anesthetic depth may be done automatically. In at least some examples, the prediction may be performed in real-time (i.e. instantaneous/immediate), or have a delay of up to 5 seconds, up to 10 seconds, up to 30 seconds, or up to 1 minute from the time the PVP waveform is received.

In some examples, the hemodynamic state predicted may be a hypervolemic state, an euvolemic state, or a hypovolemic state. In some examples, the method may predict if the patient is dehydrated or hydrated at the time of signal collection. This prediction may be useful to a physician because if a patient is dehydrated, then their veins are more constricted than if they were hydrated. The PVP waveforms may be altered by hemodynamic state as well as anesthesia.

The depth of the patient's anesthesia both in a hemorrhagic and non-hemorrhagic portion of surgery is controlled by altering the minimum alveolar concentration (MAC) of the anesthetic. The depth of a patient's anesthesia in the hemorrhagic portion of the surgery may be controlled by altering the MAC of an inhaled anesthetic. The depth in the non-hemorrhagic portion of the surgery may be controlled by applying bolus dosages of an infused anesthetic. In some examples, predicting the anesthetic depth of the patient may include predicting the patient's MAC dosage or MAC group. In various examples, the MAC dosage may be a MAC group of 1, 2, 3, 4, 5, or 6, where a higher MAC corresponds to a higher dosage of anesthetic. Predicting the MAC allows for an anesthesiologist to verify that the MAC dosage they've applied has changed the waveform exactly as intended. Also, the anesthetic depth may be assessed by the MAC that is predicted. For example, if the MAC group predicted is 3 or higher, then it is known that the patient has a high anesthetic depth. For patients receiving an infused anesthetic, the method may determine if the anesthetic is still making an effect on the waveform (e.g. 0 (no presence) or 1 (presence)).

In some examples, the prediction method may predict preoperative (i.e. the absence of anesthesia) and intraoperative signals (i.e. the presence of anesthesia) and/or may classify an arbitrary PVP signal to its correct MAC dosage or infused anesthetic bolus presence. Being able to see a significant difference in the PVP signal at different hemodynamic states has an important impact to the medical field. First, it helps the physicians to make an immediate decision in emergency situations. Also, showing a significant relationship between the anesthetic drugs, inhaled and infused, and the PVP implies that the consequent changes in vascular resistance due to the anesthetic drugs are reflected in the vein circulation and in the peripheral veins. The prediction methods herein may accurately estimate the volume status of a patient to guide triage and remediation. This may be a significant enhancement in various care settings, including but not limited to surgery, pediatrics, and military use.

The prediction method may utilize a prediction model such as a k-nearest neighbor (k-NN), neural network, random forest, SVM, naïve Bayes, and/or K-means model to predict the hemodynamic status or the anesthetic depth. The prediction model may be previously trained with anesthetic dosages and know how many separate groups of anesthetic dosages are available. Therefore, the prediction model may predict the anesthetic dosage or presence at each time point by comparing the cleaned and transformed PVP waveform to known waveforms (that were used to train the algorithm) in each anesthetic group to see which is most similar.

In some examples, the prediction method may correctly predict at least 77% of euvolemic and hypovolemic groups. The k-NN models of the anesthetic drugs may be able to correctly predict correctly at least 85% of the preoperative and intraoperative signals of the pyloric stenosis patients and the different isoflurane dosages of the craniosynostosis patients.

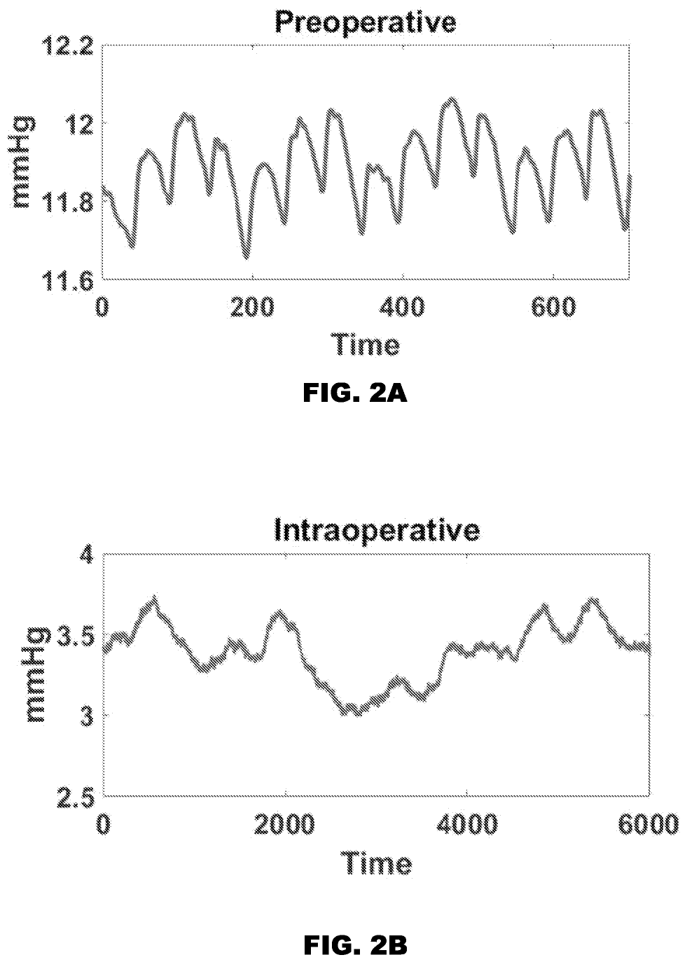

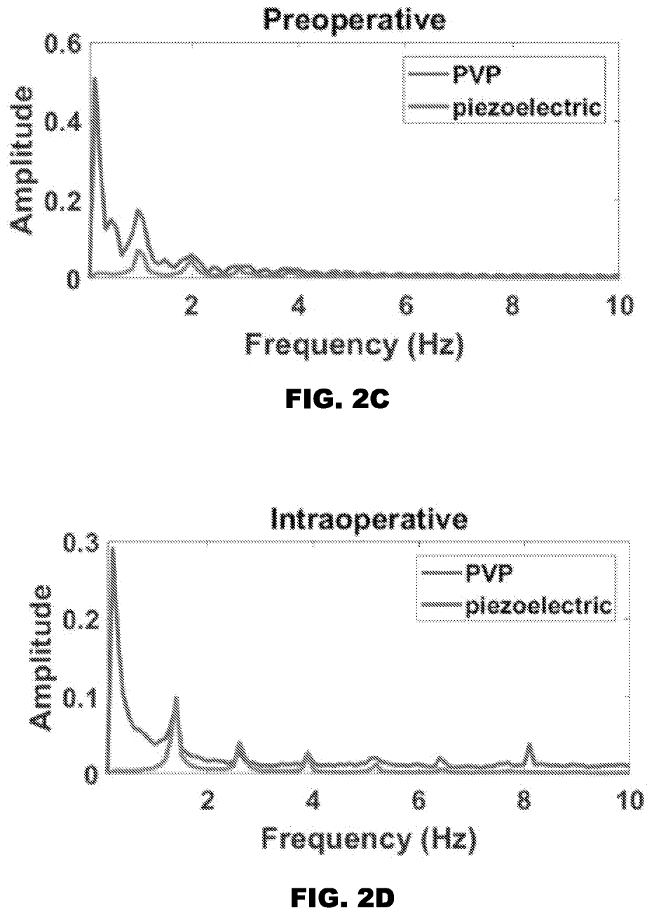

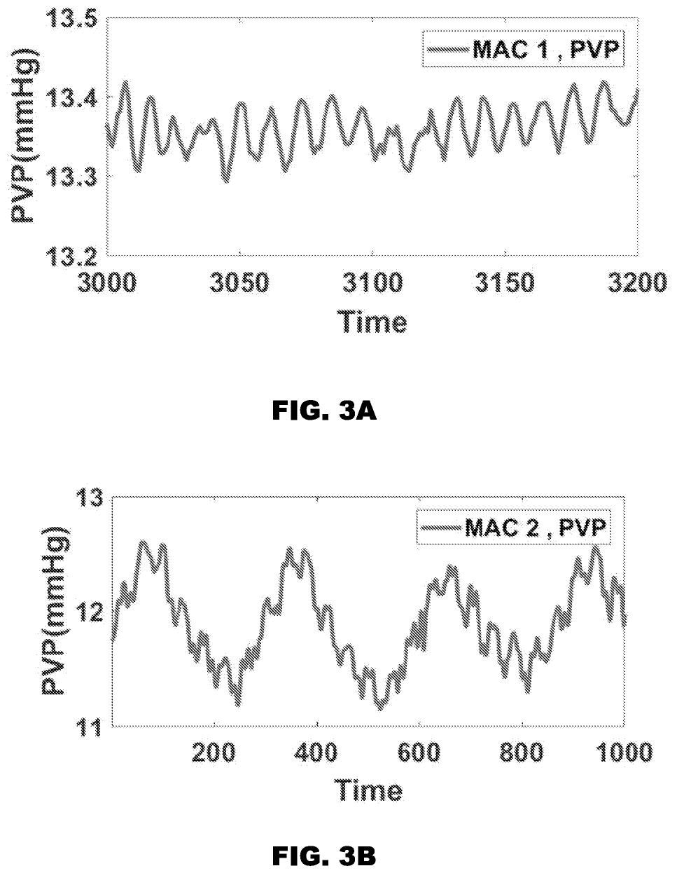

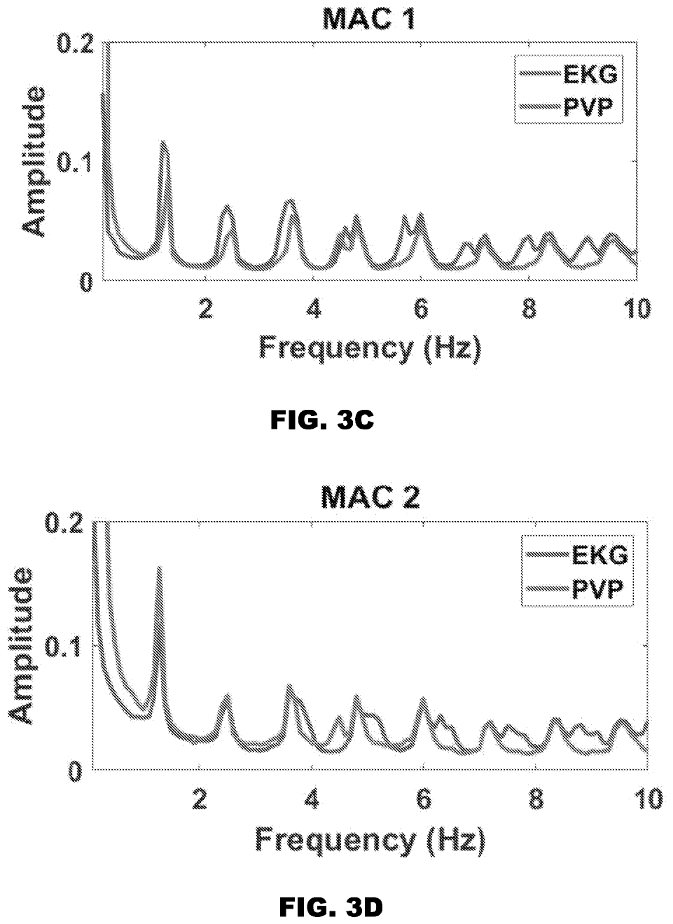

More specifically, when propofol is administered, the PVP amplitude of the intraoperative waveform decreases compared to the amplitude of the preoperative waveform. The relationship between propofol and PVP is illustrated in FIGS. 2A-2B, where the PVP amplitude in time domain is lower when propofol was introduced and the PVP harmonics follow the piezoelectric. After administering an infused anesthetic, the PVP amplitude directly decreases. The piezoelectric and PVP frequencies correlate, showing that pulse rate decreases when the patient is under anesthetics. For the isoflurane patients, whenever MAC increases, the PVP waveform decreases. This demonstrates that increasing MAC immediately dilates the veins and reduces venous pressure; the relationship is illustrated in FIGS. 3A-3B. FIGS. 3A-3D show the PVP amplitude in time domain is lower in higher MAC dosages and the PVP harmonics follow the patient's electrocardiography (ECG/EKG).

In additional examples, the method may further include measuring the patient's ECG. The method may also include determining ECG and PVP waveform coefficients at the heart rate and respiratory rate frequencies. Measuring the ECG along with the PVP may identify the frequency that corresponds to the heart rate and whether it is matching the frequency at the highest peak of the PVP waveform. There is a robust mimicking between the frequency of PVP and the frequency of ECG and the frequencies at the highest amplitude in FIGS. 2C-2D are equal, 1.2 Hz. In human arms and legs, peripheral arteries and veins run in close anatomical proximity, and it is feasible to assume that the pressure in one vessel can carry over to the other. Without being limited to any particular theory, it appears that in hydrated patients, the cross-talk between arteries and veins in direct physical interaction with each other accounts for the signal waveform in frequencies corresponding to heart rate. When the patient has adequate blood volume, the arterial pulse pressure waveform crosses over to the venous side. In dehydrated patients, as the diameter of arteries and veins decreases, the cross-talk is lost and the signal waveform is affected at the frequency of the heart rate. Therefore, the methods herein may take into account the heart rate of a patient, to prevent the limitation of PVP signal analysis.

In some examples, the method may further include preventing overdosage or underdosage of anesthesia during a medical operation. In at least one example, the medical may be a pediatric medical operation. The automatic prediction of the hemodynamic status or anesthetic depth in the patient may inform a physician of how adjust or correct the dosage of anesthesia being administered to the patient to prevent overdosage or underdosage. For example, a minimum and/or maximum anesthetic depth may be provided by the physician or may be pre-set. Then, the dosage administered to the patient may be adjusted to maintain the predicted anesthetic depth within the minimum and maximum values to prevent overdosage or underdosage. The dosage being administered to the patient may be adjusted automatically or may be adjusted manually by the physician.

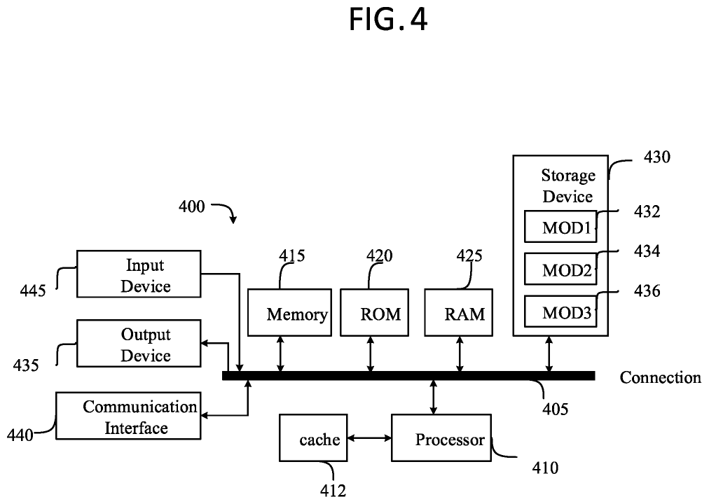

The disclosure now turns to the example system illustrated in FIG. 4 which may be used to implement the methods for predicting a hemodynamic state and/or anesthetic depth of a patient. In an example, a device may include a computing system having at least one processor for predicting a patient's hemodynamic status and/or anesthetic depth. FIG. 4 shows an example of computing system 400 in which the components of the system are in communication with each other using connection 405. Connection 405 can be a physical connection via a bus, or a direct connection into processor 410, such as in a chipset or system-on-chip architecture. Connection 405 can also be a virtual connection, networked connection, or logical connection.

In some examples computing system 400 is a distributed system in which the functions described in this disclosure can be distributed within a datacenter, multiple datacenters, a peer network, throughout layers of a fog network, etc. In some examples, one or more of the described system components represents many such components each performing some or all of the function for which the component is described. In some examples, the components can be physical or virtual devices.

Example system 400 includes at least one processing unit (CPU or processor) 410 and connection 405 that couples various system components including system memory 415, read only memory (ROM) 420 or random access memory (RAM) 425 to processor 410. Computing system 400 can include a cache of high-speed memory 412 connected directly with, in close proximity to, or integrated as part of processor 410.

Processor 410 can include any general purpose processor and a hardware service or software service, such as services 432, 434, and 436 stored in storage device 430, configured to control processor 410 as well as a special-purpose processor where software instructions are incorporated into the actual processor design. Processor 410 may essentially be a completely self-contained computing system, containing multiple cores or processors, a bus, memory controller, cache, etc. A multi-core processor may be symmetric or asymmetric.

To enable user interaction, computing system 400 includes an input device 445, which can represent any number of input mechanisms, such as a microphone for speech, a touch-sensitive screen for gesture or graphical input, keyboard, mouse, motion input, speech, etc. Computing system 400 can also include output device 435, which can be one or more of a number of output mechanisms known to those of skill in the art. In some instances, multimodal systems can enable a user to provide multiple types of input/output to communicate with computing system 400. Computing system 400 can include communications interface 440, which can generally govern and manage the user input and system output, and also connect computing system 400 to other nodes in a network. There is no restriction on operating on any particular hardware arrangement and therefore the basic features here may easily be substituted for improved hardware or firmware arrangements as they are developed.

Storage device 430 can be a non-volatile memory device and can be a hard disk or other types of computer readable media which can store data that are accessible by a computer, such as magnetic cassettes, flash memory cards, solid state memory devices, digital versatile disks, cartridges, battery backed random access memories (RAMs), read only memory (ROM), and/or some combination of these devices.

The storage device 430 can include software services, servers, services, etc., that when the code that defines such software is executed by the processor 410, it causes the system to perform a function. In some examples, a hardware service that performs a particular function can include the software component stored in a computer-readable medium in connection with the necessary hardware components, such as processor 410, connection 405, output device 435, etc., to carry out the function.

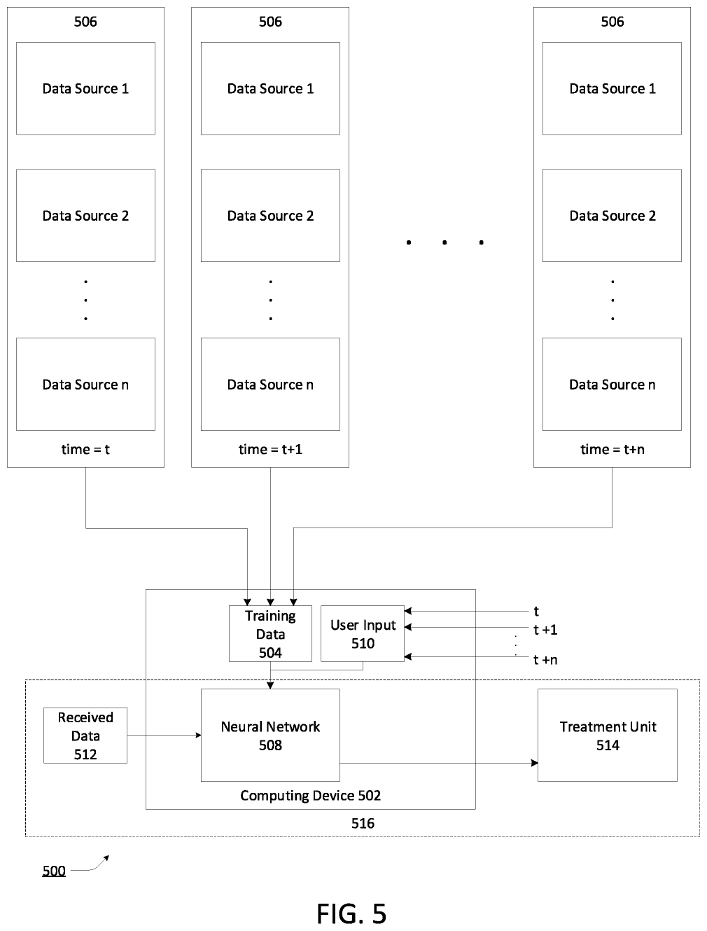

The disclosure now turns to FIG. 5, which illustrates an example machine learning environment 500. The machine learning environment can be implemented on one or more computing devices 502A-N (e.g., cloud computing servers, virtual services, distributed computing, one or more servers, etc.). The computing device(s) 502 can include training data 504 (e.g., one or more databases or data storage device, including cloud-based storage, storage networks, local storage, etc.). In some examples, the training data may include data from patients that have undergone a pyloromyotomy or craniosynostosis surgery with an infused or inhaled anesthetic. The training data 504 of the computing device 502 can be populated by one or more data sources 506 (e.g., data source 1, data source 2, data source n, etc.) over a period of time (e.g., t, t+1, t+n, etc.). In some examples, training data 504 can be labeled data (e.g., one or more tags associated with the data). For example, training data can be one or more PVP waveforms and a label (e.g., MAC value, hemodynamic status, etc.) can be associated with each waveform. The computing device(s) 502 can continue to receive data from the one or more data sources 506 until the neural network 508 (e.g., convolution neural networks, deep convolution neural networks, artificial neural networks, learning algorithms, etc.) of the computing device(s) 502 are trained (e.g., have had sufficient unbiased data to respond to new incoming data requests and provided an autonomous or near autonomous image classification). In some examples, the neural network can be a convolutional neural network, for example, utilizing five layer blocks, including convolutional blocks, convolutional layers, and fully connected layers. In some examples, the neural network may utilize a k-nearest neighbor, neural network, random forest, SVM, naïve Bayes, and/or K-means model. While example neural networks are realized, neural network 508 can be one or more neural networks of various types are not specifically limited to a single type of neural network or learning algorithm.

In other examples, a feature selection can be generated (e.g., group correlated features such that one feature is used for each group). In these instances, cleaned and transformed segments of a PVP waveform are used in a prediction model. The training data can require a minimum or an equivalent number of PVP waveform segments per patient.

In some examples, while not shown here, the training data 504 can be checked for biases, for example, by checking the data source 506 (and corresponding user input) verse previously known unbiased data. Other techniques for checking data biases are also realized. The data sources can be any of the sources of data for providing the PVP waveforms (e.g., IV pressure transducer, etc.) as described above in this disclosure.

The computing device(s) 502 can receive user (e.g., physician) input 510 related to the data source. The user input 510 and the data source 506 can be temporally related (e.g., by time t, t+1, t+n, etc.). That is, the user input 510 and the data sources 506 can be synchronous in that the user input 510 corresponds and supplements the data source 506 in a manner of supervised or reinforced learning. For example, a data source 506 can provide a PVP waveform at time t and corresponding user input 510 can be input of hemodynamic status or MAC group of that PVP waveform at time t. While, time t may actually be different in real-world time, they are synchronized in time with respect to the data provided to the training data.

The training data 504 can be used to train a neural network 508 or learning algorithms (e.g., convolutional neural network, artificial neural network, etc.). The neural network 508 can be trained, over a period of time, to automatically (e.g., autonomously) determine what the user input 510 would be, based only on received data 512 (e.g., PVP waveform, etc.). For example, by receiving a plurality of unbiased data and/or corresponding user input for a long enough period of time, the neural network will then be able to determine what the user input would be when provided with only the data. For example, a trained neural network 508 will be able to receive a PVP waveform (e.g., 512) and based on the PVP waveform determine the hemodynamic status or anesthetic depth that a physician would manually identify (and that would have been provided as user input 510 during training). In some examples, this can be based on labels associated with the data as described above. The output from the trained neural network can be provided to a prediction model 514 for treating a patient. In some examples, the output from the trained neural network can be inputted directly into a prediction model to predict a hemodynamic status and/or anesthetic depth in the patient.

Trained neural network system 516 can include a trained neural network 508, received data 512, and prediction model 514. The received data 512 can be information related to a patient, as previously described above. The received data 512 can be used as input to trained neural network 508. Trained neural network 508 can then, based on the received data 512, label the received data and/or determine a recommended course of action for treating the patient, based on how the neural network was trained (as described above). The recommended course of action or output of trained neural network 508 can be used as an input into the prediction model 514 (e.g., to predict the hemodynamic status and/or anesthetic depth for the patient to which the received data 512 corresponds). In other instances, the output from the trained neural network can be provided in a human readable form, for example, to be reviewed by a physician to determine a course of action.

For clarity of explanation, in some instances the present technology may be presented as including individual functional blocks including functional blocks comprising devices, device components, steps or routines in a method embodied in software, or combinations of hardware and software.

In some embodiments the computer-readable storage devices, mediums, and memories can include a cable or wireless signal containing a bit stream and the like. However, when mentioned, non-transitory computer-readable storage media expressly exclude media such as energy, carrier signals, electromagnetic waves, and signals per se.

Methods according to the above-described examples can be implemented using computer-executable instructions that are stored or otherwise available from computer readable media. Such instructions can comprise, for example, instructions and data which cause or otherwise configure a general purpose computer, special purpose computer, or special purpose processing device to perform a certain function or group of functions. Portions of computer resources used can be accessible over a network. The computer executable instructions may be, for example, binaries, intermediate format instructions such as assembly language, firmware, or source code. Examples of computer-readable media that may be used to store instructions, information used, and/or information created during methods according to described examples include magnetic or optical disks, flash memory, USB devices provided with non-volatile memory, networked storage devices, and so on.

Devices implementing methods according to these disclosures can comprise hardware, firmware and/or software, and can take any of a variety of form factors. Typical examples of such form factors include laptops, smart phones, small form factor personal computers, personal digital assistants, rackmount devices, standalone devices, and so on. Functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are means for providing the functions described in these disclosures.

### EXAMPLES

### Example 1: Acquiring PVP

The impact of anesthetics on PVP waveforms was tested in two anesthetized patient cohorts. The first cohort represented a dehydration setting in infants operated on for pyloric stenosis diagnosed by ultrasound who had been projectile vomiting and admitted prior to undergoing a pyloromyotomy operation during which propofol was infused as an anesthetic. Data was collected after being resuscitated to near euvolemia at the time of operation. The second cohort represented a hemorrhagic setting in infants operated on during a reconstructive, elective craniosynostosis operation.

Due to the vast blood supply to the skull, intra-operative estimated blood loss of 60-70 cc/kg and occasionally up to half of blood volume may need to be replaced utilizing a combination of intravenous fluids (IVF), blood products, and occasionally pressors.

These two cohorts were utilized to determine if anesthetics such as propofol or isoflurane influenced the PVP waveform. After determining the relationship, two machine learning systems were built using a k-nearest neighbor statistical model to predict hydration levels for arbitrary pyloric stenosis PVP waveforms, and also predict MAC for an arbitrary craniosynostosis PVP waveform.

PVP waveforms were collected from 39 pyloric stenosis patients and 9 craniosynostosis patients. For the pyloric stenosis patients, three patients were removed because a Nexiva catheter was used instead of the PIV catheter, resulting in a distinctly different PVP waveform. Two other patients were discarded because their PIV catheters were inserted into the foot. Eleven patients were excluded due to either a flat PVP waveform due to incorrect zeroing of catheter or other circumstances that rendered the data unusable. This resulted in a total of twenty-three patients used for waveform analysis. The patients were further sorted based on their hydrations status when they arrived at the emergency room, either hypovolemic with severe fluid loss, or euvolemic with normal fluid volume. Statistical testing for hypovolemic patients and euvolemic patients were conducted separately. For the isoflurane testing, nine patients were initially included in the study. Two patients were removed because the time of the operation start was not noted when LabChart started recording the PVP, making it difficult to relate MAC and PVP. The seven isoflurane patients were further sorted, based on the number of MAC groups used during the operation. For each patient, there were n MAC groups that were assigned a group number n>0 when MAC fell between n−1 and n−0.1. For example, if MAC ranged between [0-0.9], then it would be classified as MAC group 1.

The average weight of the fifteen enrolled euvolemic pyloric stenosis pediatric patients was 4.14 kilograms (kg) with a standard deviation of 0.68 kg. The average weight of the eight hypovolemic patients was 3.70 kg with a standard deviation of 0.74 kg, which was lower than the euvolemic patients. After enrollment, fluids were given to the hypovolemic patients so that at the time of the operation, the twenty-three patients were all considered euvolemic. The average weight of the enrolled craniosynostosis pediatric patients was 10 kg with a standard deviation of 3.66 kg.

For the pyloric stenosis patients, data points were collected over the entire operation, and for the craniosynostosis patients, data points were collected from the first instance of isoflurane throughout the procedure until isoflurane administration was ceased. PVP waveforms were measured with a 24-gauge Insyte-N Autoguard peripheral intravenous (PIV) catheter. The PIV catheter was connected to a Deltran II pressure transducer using 48-inch arterial pressure tubing. Then, a Powerlab data acquisition system (ADInstruments) was used to connect the hardware setup with LabChart 8 (AD Instruments) to record the waveforms.

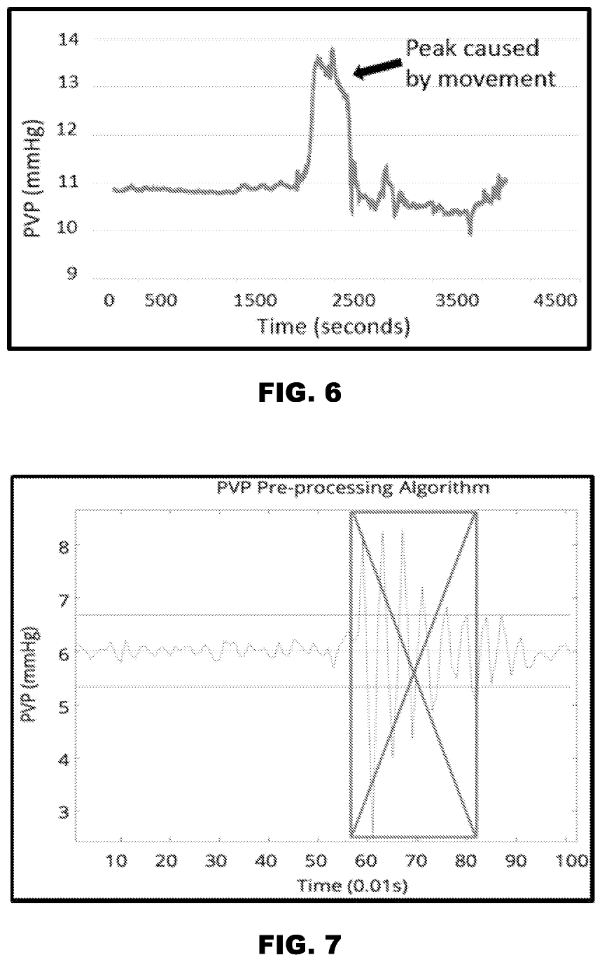

The Deltran pressure transducer detects small movements of the infant, bed movement, infant's crying, or apparatus errors which interferes with the PVP recording. Movement causes large spikes in the recorded waveform as shown in FIG. 6. Other external factors can potentially interfere with the PVP measuring accuracy, such as adjusting the tubing or accidentally hitting the operative table.

### Example 2: Data Cleaning Algorithm and Fast Fourier Transform

Due to waveform contamination due to undesired artifacts mentioned in Example 1, an algorithm was developed using MATLAB to pre-process the data and remove the unwanted sections of the waveforms.

First, the entire PVP waveform was sampled at a rate of 100 Hz from LabChart 8 for each patient. After sampling the waveform, the PVP data was exported into a custom algorithm. For isoflurane patients, the corresponding MAC values were exported alongside the corresponding PVP waveforms. The algorithm takes sections of the PVP data at a user-selected length of time to analyze. The algorithm calculates the remainder of the PVP signal divided by pre-selected time length, the length of the segment, and then remove the last points of the signal that are equal to the PVP signal remainder. These two steps assure that every single segment has the same duration for all the patients. For every section of the PVP waveform signal, the mean value of the data values in that section was calculated, and if any data points in that time section exceeds above or below the user-defined number of standard deviations, then the entire section of data is removed; this method is illustrated in FIG. 7. The algorithm goes through the entire PVP waveform, which can be up to 4 hours long for the isoflurane patients, and removes sections of the data that contain spikes within the segments due to movement. The process takes a maximum of two minutes.

### Example 3: Fast Fourier Transform

Each segment of the PVP signal was transformed into the frequency domain using a Fast Fourier Transform (FFT) function. The analyses were in the frequency domain because it reduces the cost and time of the testing and it is more stable because of the absence of the negative feedback. Also, frequency domain is used to check the dominant amplitudes that reflects many factors such as the heart pulse and respiratory rate.

After the cleaning algorithm, the data was divided into 10-second windows. Each window contains only a continuous waveform, that is, if a section of the waveform was removed during the cleaning process, the waveforms before and after the removed section will not be in the same window. Thus, the frequency domain resolution was 0.1 Hz which represents the distance between two frequency samples. With a time domain sampling rate of 100 Hz, the signal covered a frequency range of 50 Hz. However, only signals from 0 to 20 Hz were used for further processing. When converting the data to the frequency domain, the result is two mirrored values at different frequencies, so using the first 20 Hz ensures that the used bins do not belong to the same frequencies. Furthermore, there is no useful information after the 20th bins since no one can have a heart rate that is greater than 20 Hz. Thus, the total number of bins was 200 and each bin was a feature of the PVP signal at different frequency with 0.1 step frequency size. However, the 200 features were down sampled by a factor of 4 leading to have a 0.4 step frequency size with 50 points for each 10-second segment. The down sampling ensures that the number of observations is more than the number of variables to get reliable results because having 200 frequency features may not be fulfilled in some recorded PVP waveforms due to the small number of observations, less than 200.

### Example 4: Statistical Analysis

During the pyloromyotomy surgery, the patients received propofol. In order to test if the propofol influences the PVP, the intraoperative PVP signal was tested against the preoperative PVP signal when the patient had not received any propofol. It was tested was if the intraoperative and the preoperative PVP waveforms were significantly different; MANOVA was used to test the hypothesis.

The data presented for the isoflurane patients contains a continuous PVP measurement during the craniosynostosis operation while the MAC dosage is changing over time. Linear regression and MANOVA were used to test if PVP signal is influenced by MAC.

The linear regression model fit requires the input and the output to be continuous to examine the data linearity. One of the parameters to look at in linear regression is the coefficient of determination (R-squared) which measures how close the fitted line is to the data. As R-squared increases, the model shows a more linear relationship between the two continuous variables.

Rstudio was used to perform the multivariate analysis of variance (MANOVA) test. For the MANOVA test, a significance level of 0.05 was used. The Pillai's trace was the chosen test statistic due to its robustness. For the propofol waveforms, the independent variable was the classification number that was assigned to the intraoperative and preoperative PVP signals and the dependent variable was the PVP waveform. For the isoflurane waveforms, the independent variable was the MAC group, and the dependent variable was the PVP waveform.

Pairwise MANOVA was also applied for all groups of data collected from both the propofol and isoflurane data to ensure the results were reliable and are shown in Table 1.

TABLE 1
MANOVA pairwise of isoflurane patients

| | |
|---|---|
| Group 1 | [0-0.9] |
| Group 2 | [1-1.9] |
| Group 3 | [2-2.9] |
| Group 4 | [3-3.9] |

The null hypothesis in the craniosynostosis cohort patients is that as MAC dosage changes, there is no significant influence on the PVP signal. On the other hand, the alternative hypothesis states that the PVP waveform significantly changes as MAC dosage varies. For the patients whose MAC dosages were categorized into more than two MAC groups, a MANOVA pairwise test was needed to check which groups are different and which groups are the same.

The MANOVA p-values and the Pillai's trace were calculated and are shown in Tables 2 and 3 below.

TABLE 2
MANOVA results for propofol study.

| | | df | | Partial | p- |
|---|---|---|---|---|---|
| | df | error | F | h² | value |
| Hypovolemia | 50 | 327 | 6.0 | 0.478 | <0.01 |
| Euvolemia | 50 | 302 | 3.8 | 0.388 | <0.01 |

TABLE 3
MANOVA results for isoflurane study.

| Patient | | df | | Partial | p- |
|---|---|---|---|---|---|
| # | df | error | F | h² | value |
| 3 | 50 | 231 | 4.6 | 0.499 | <0.01 |
| 4 | 50 | 221 | 3.0 | 0.406 | <0.01 |
| 5 | 50 | 249 | 2.8 | 0.359 | <0.01 |
| 6 | 50 | 571 | 17.3 | 0.602 | <0.01 |
| 7 | 50 | 101 | 2.9 | 0.586 | <0.01 |
| 8 | 50 | 110 | 7.3 | 0.768 | <0.01 |
| 9 | 50 | 226 | 3.7 | 0.450 | <0.01 |

The results in the previous two tables show a significant relationship between the PVP signal and the effect of anesthetics.

### Example 5: Machine Learning Algorithms

MATLAB was used to develop k-nearest neighbor (k-NN) statistical models and build machine learning prediction systems for the propofol and isoflurane PVP waveform.

Prediction models were designed using k-nearest neighbor (k-NN) (k=1) for creating the machine learning prediction systems for the propofol and isoflurane patients. For both the propofol and isoflurane studies, 70% of the data were used for training and the remaining 30% were used for testing. However, the model parameters, /3, are unknown, and are being calculated. The training data is used to calculate the /3 coefficients and then the validation data is used to test if those calculated parameters are reliable to predict the output of the testing data correctly. Results from the machine learning systems are shown in Tables 4-9 below.

The k-nearest neighbor (k-NN) algorithm was able to classify 94 data points out of 122, 77%, for the testing data of the hypovolemic group. For the euvolemic group, the k-NN model was able to predict correctly 38 data points out of 50, 76%. Also, the algorithm was able to predict 243 data points out of 285, 85%, for the training data of the hypovolemic group and 100 data points out of 118, 85%, of the euvolemic group (Table 5). Being able to predict the class of an arbitrary PVP indicates that any volume change in the body state is detectable by the peripheral veins and machine learning can be implemented to predict the intravascular volume status of future patients without having any further information about the patient's medical record.

TABLE 4
K-NN prediction results for propofol
study out of the total number of windows.

| | Correct | Incorrect |
|---|---|---|
| | Prediction | Prediction |
| Hypovolemia | 96/106 | 10/106 |
| Euvolemia | 102/114 | 12/114 |

TABLE 5
Confusion matrix using k-nearest neighbor

| Testing data | | | Training Data | | |
|---|---|---|---|---|---|
| | Hypovol | Euvol | | Hypovol | Euvol |
| Hypovol | 94 | 28 | Hypovol | 243 | 42 |
| Euvolem | 12 | 38 | Euvolem | 18 | 100 |

The k-NN model was able to predict 78 windows out of 81, 96%, of the Preop signal for the hypovolemic group. On the other hand, the model was able to classify 20 windows out of 23, 87%, of the OR signal correctly. Also, the k-NN model was able to predict 115 out of 118, 97%, and 43 out of 54, 80%, for the training data of the Preop and OR signals, respectively (Table 6). Therefore, these results indicate that machine learning can be used to predict the volume status of future patients using only the PVP signal without the need to know the patient's medical records.

TABLE 6
Hypovolemic group confusion
matrix using k-nearest neighbor

| Testing data | | | Training Data | | |
|---|---|---|---|---|---|
| | Preop | OR | | Preop | OR |
| Preop | 78 | 3 | Preop | 115 | 3 |
| OR | 3 | 20 | OR | 11 | 43 |

The k-NN model was able to predict 96 windows out of 109, 88%, of the Preop signal for the euvolemic group. Likewise, the k-NN model predicted 37 windows correctly out of 45, 82%, of the OR signal (Table 7).

TABLE 7
Euvolemic group confusion
matrix using k-nearest neighbor

| Testing data | | | Training Data | | |
|---|---|---|---|---|---|
| | Preop | OR | | Preop | OR |
| Preop | 96 | 13 | Preop | 244 | 11 |
| OR | 8 | 37 | OR | 25 | 80 |

The correct and mismatch predictions at different isoflurane dosages for the testing and training data using k-NN are in Tables 8 and 9. The results illustrate that the change in vascular resistance is detectable in the venous circulation and the PVP signal. The machine learning system was able to accurately distinguish between the PVP waveforms of each MAC group and predict the correct MAC classification for an arbitrary PVP at least 77% of the time.

TABLE 8
K-NN prediction results for isoflurane
study out of the total number of windows.

| | Correct | Incorrect |
|---|---|---|
| Patient # | Prediction | Prediction |
| 3 | 66/82 | 16/82 |
| 4 | 55/67 | 12/67 |
| 5 | 89/90 | 1/90 |
| 6 | 143/186 | 43/186 |
| 7 | 27/35 | 8/35 |
| 8 | 23/28 | 5/28 |
| 9 | 64/82 | 18/82 |

TABLE 9
Confusion matrices of k-NN algorithm

| Patient # | Testing data | | | Training Data | | |
|---|---|---|---|---|---|---|
| 3 | | MAC 1 | MAC 2 | | MAC 1 | MAC 2 |
| | MAC 1 | 60 | 10 | MAC 1 | 164 | 0 |
| | MAC 2 | 6 | 6 | MAC 2 | 0 | 29 |
| 4 | | MAC 1 | MAC 2 | | MAC 1 | MAC 2 |
| | MAC 1 | 28 | 8 | Group 1 | 83 | 0 |
| | MAC 2 | 4 | 27 | Group 2 | 0 | 74 |
| 5 | | MAC 1 | MAC 2 | | MAC 1 | MAC 2 |
| | MAC 1 | 0 | 1 | MAC 1 | 1 | 0 |
| | MAC 2 | 0 | 89 | MAC 2 | 0 | 209 |
| 6 | | MAC 1 | MAC 2 | | MAC 1 | MAC 2 |
| | MAC 1 | 97 | 3 | MAC 1 | 234 | 0 |
| | MAC 2 | 8 | 78 | MAC 2 | 0 | 202 |
| 7 | | MAC 1 | MAC 2 | | MAC 1 | MAC 2 |
| | MAC 1 | 17 | 3 | MAC 1 | 48 | 0 |
| | MAC 2 | 5 | 10 | MAC 2 | 0 | 35 |
| 8 | | MAC 1 | MAC 2 | | MAC 1 | MAC 2 |
| | MAC 1 | 1 | 3 | MAC 1 | 10 | 0 |
| | MAC 2 | 2 | 22 | MAC 2 | 0 | 57 |
| 9 | | MAC 1 | MAC 2 | | MAC 1 | MAC 2 |
| | MAC 1 | 25 | 12 | MAC 1 | 87 | 0 |
| | MAC 2 | 6 | 39 | MAC 2 | 0 | 104 |

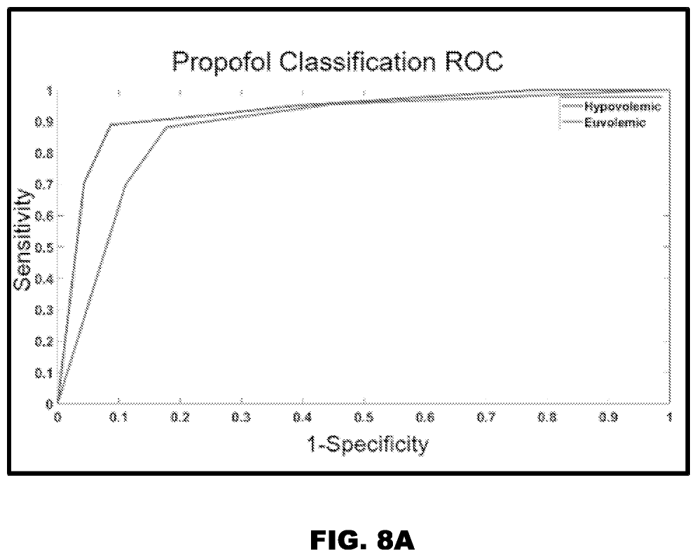

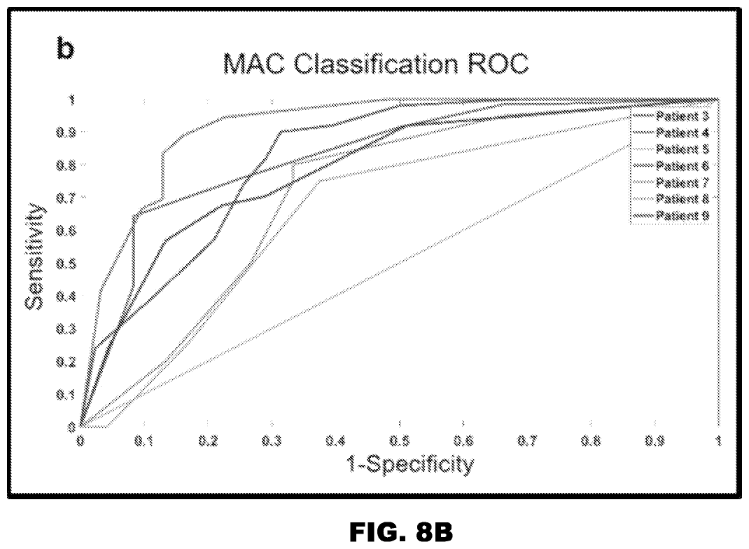

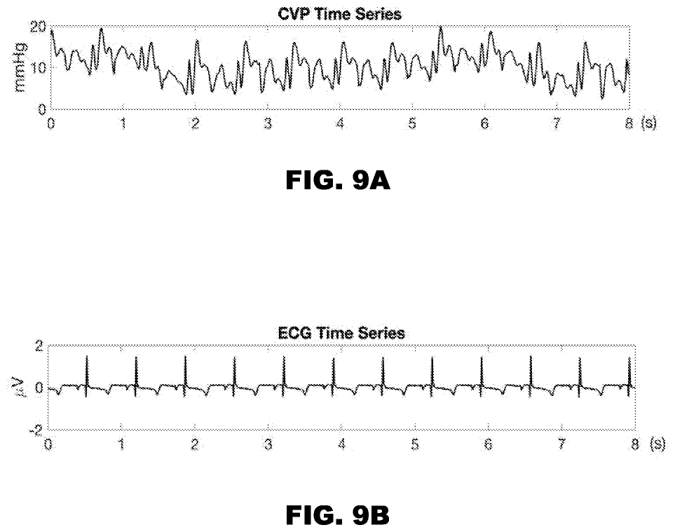

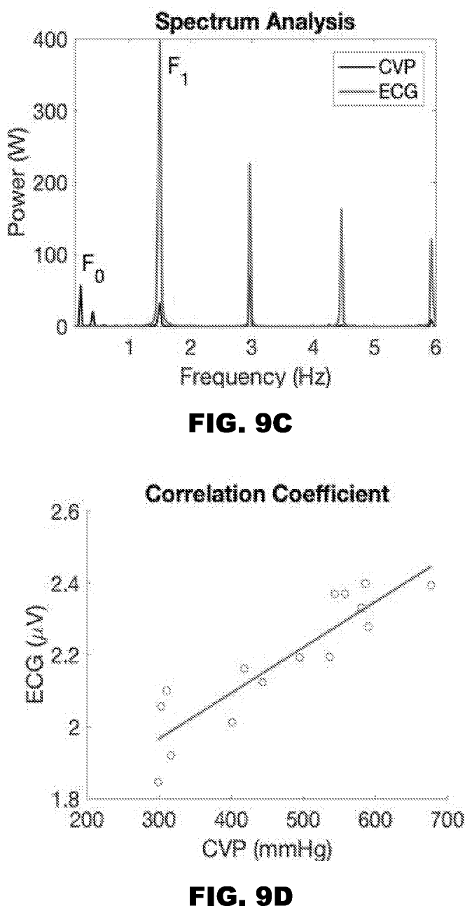

The previous tables show the number of data points that the machine learning algorithm predicted correctly for the propofol and the isoflurane. A receiver operating characteristic (ROC) curve was plotted for each cohort to illustrate the machine learning model's ability to classify the data and is shown in FIGS. 8A-8B. ROC is plotted as 1-specificity vs sensitivity, with 1-specificity=|FP|/(|FP|+|TN|) and sensitivity=|TP|/(|TP|+|FN|), where FP is false positive, FN is false negative, TP is true positive, and TN is true negative.

In addition to identifying a relationship between PVP waveforms and anesthetics, a machine learning prediction model can distinguish between PVP waveforms that have propofol and those that have no anesthetics with at least 89% accuracy, as displayed in Table 4. The ROC curve in FIG. 8A has a high area under the curve for both the hypovolemic and euvolemic data, which illustrates a high-performance measure for the machine learning model. The machine learning prediction model for the isoflurane patients accurately distinguishes between the MAC groups in each patient's PVP waveform at least 77% of the time, shown in Table 8. The ROC curve in FIG. 8B shows the highest area under the curve for patient 4, so the model has the best performance for that patient. The curves for patients 3, 6, 7, 8, and 9 show that the model is performing well at predicting the MAC groups but fails to perform for patient 5. This may be due to patient 5 having a smaller amount of clean PVP data to analyze or insufficient training data for each of the MAC groups specific to the patient. Overall, these high correct prediction results further support the conclusion that anesthetics affect the PVP waveform.

Two additional prediction models were also tested, a logistic regression and LASSO regression model. The difference between logistic regression and LASSO regression is that the former takes all frequencies into account, even if some of them are not dominant. On the other hand, LASSO regression, which is a selection model tool, sets those unimportant parameters to zero. Therefore, the LASSO model provides a better performance with as small prediction error as possible.

The LASSO algorithm predicted correctly all the testing and training data for the hypovolemic group whereas the LASSO model did not predict correctly any data for the euvolemic group (Table 10).

TABLE 10
Confusion matrix using LASSO regression

| Testing data | | | Training Data | | |
|---|---|---|---|---|---|
| | Hypo- | Eu- | | Hypo- | Eu- |
| | volemic | volemic | | volemic | volemic |
| Hypo- | 122 | 0 | Hypo- | 285 | 0 |
| volemic | | | volemic | | |
| Eu- | 50 | 0 | Eu- | 118 | 0 |
| volemic | | | volemic | | |

The logistic regression algorithm predicted correctly 109 out of 122 for the testing data of the hypovolemic group whereas 11 were correctly predicted out of 50 for the euvolemic group. The training data was used as an input to the logistic regression system to check if the machine learning model is able to predict the data that was originally used to train the model. The algorithm predicted correctly 260 out of 285 for the training data of the hypovolemic group whereas 76 data points out of 118 were correctly predicted for the euvolemic group (Table 11).

TABLE 11
Confusion matrix using logistic regression

| Testing data | | | Training Data | | |
|---|---|---|---|---|---|
| | Hypo- | Eu- | | Hypo- | Eu- |
| | volemic | volemic | | volemic | volemic |
| Hypo- | 109 | 13 | Hypo- | 260 | 25 |
| volemic | | | volemic | | |
| Eu- | 39 | 11 | Eu- | 76 | 42 |
| volemic | | | volemic | | |

The logistic regression model was able to predict all the data points, testing and training data, correctly for the preoperative (preop) signal of the hypovolemic group. However, the prediction accuracy for the intraoperative (OR) signal for the testing and the training data was 0% (Table 12).

TABLE 12
Hypovolemic group confusion
matrix using logistic regression

| Testing data | | | Training Data | | |
|---|---|---|---|---|---|
| | Preop | OR | | Preop | OR |
| Preop | 81 | 0 | Preop | 188 | 0 |
| OR | 23 | 0 | OR | 54 | 0 |

For the testing data of the euvolemic group, the preoperative signal had 91% prediction accuracy, whereas, the intraoperative prediction accuracy was approximately 50%. For the training data, the preoperative was able to predict 250 data points correctly out of 255, 98%. Whereas 91 data points out of 105 were predicted correctly for the intraoperative signal, 87% (Table 13).

TABLE 13
Euvolemic group confusion
matrix using logistic regression

| Testing data | | | Training Data | | |
|---|---|---|---|---|---|
| | Preop | OR | | Preop | OR |
| Preop | 99 | 10 | Preop | 250 | 5 |
| OR | 21 | 24 | OR | 14 | 91 |

The LASSO regression model of the hypovolemic and euvolemic groups was able to predict correctly all the Preop data points for the training and testing data. However, the LASSO algorithm failed to predict correctly any data points of the testing and training data of the euvolemic and hypovolemic groups for the OR signal (Table 14 and 15).

TABLE 14
Hypovolemic group confusion
matrix using LASSO regression

| Testing data | | | Training Data | | |
|---|---|---|---|---|---|
| | Preop | OR | | Preop | OR |
| Preop | 81 | 0 | Preop | 188 | 0 |
| OR | 23 | 0 | OR | 54 | 0 |

TABLE 15
Euvolemic group confusion
matrix using LASSO regression

| Testing data | | | Training Data | | |
|---|---|---|---|---|---|
| | Preop | OR | | Preop | OR |
| Preop | 109 | 0 | Preop | 255 | 0 |
| OR | 45 | 0 | OR | 105 | 0 |

The linear regression and multiple logistic regression results for the craniosynostosis patients are in Tables 16 and 17. The R-squared values for all the patients are in Table 16 and the mean absolute error of linear regression was calculated and listed in Table 17.

TABLE 16
R-squared for the
linear regression of the
craniosynostosis patients

| Patient # | |
|---|---|
| 3 | 0.583 |
| 4 | 0.387 |
| 5 | 0.329 |
| 6 | 0.634 |
| 7 | 0.565 |
| 8 | 0.784 |
| 9 | 0.512 |

TABLE 17
Mean absolute error of
linear regression for the
craniosynostosis patients

| Patient | Linear |
|---|---|
| # | Regression |
| 3 | 17.38% |
| 4 | 44.33% |
| 5 | 3.09% |
| 6 | 9.06% |
| 7 | 14.88% |
| 8 | 19.06% |
| 9 | 13.58% |

The correct and mismatch predictions at different isoflurane dosages for the testing and training data using multiple logistic regression are in Table 18.

TABLE 18
Confusion matrices of craniosynostosis
using multiple logistic regression

| Patient # | Testing data | | | Training Data | | |
|---|---|---|---|---|---|---|
| 3 | | MAC 1 | MAC 2 | | MAC 1 | MAC 2 |
| | MAC 1 | 60 | 10 | MAC 1 | 163 | 1 |
| | MAC 2 | 6 | 6 | MAC 2 | 12 | 17 |
| 4 | | MAC 1 | MAC 2 | | MAC 1 | MAC 2 |
| | MAC 1 | 29 | 7 | MAC 1 | 83 | 0 |
| | MAC 2 | 13 | 18 | MAC 2 | 11 | 63 |
| 5 | | MAC 1 | MAC 2 | | MAC 1 | MAC 2 |
| | MAC 1 | 0 | 1 | MAC 1 | 1 | 0 |
| | MAC 2 | 0 | 89 | MAC 2 | 0 | 209 |
| 6 | | MAC 1 | MAC 2 | | MAC 1 | MAC 2 |
| | MAC 1 | 93 | 7 | MAC 1 | 225 | 9 |
| | MAC 2 | 8 | 78 | MAC 2 | 9 | 193 |
| 7 | | MAC 1 | MAC 2 | | MAC 1 | MAC 2 |
| | MAC 1 | 14 | 6 | MAC 1 | 48 | 0 |
| | MAC 2 | 5 | 10 | MAC 2 | 0 | 35 |
| 8 | | MAC 1 | MAC 2 | | MAC 1 | MAC 2 |
| | MAC 1 | 1 | 3 | MAC 1 | 10 | 0 |
| | MAC 2 | 3 | 21 | MAC 2 | 0 | 57 |
| 9 | | MAC 1 | MAC 2 | | MAC 1 | MAC 2 |
| | MAC 1 | 24 | 13 | MAC 1 | 74 | 13 |
| | MAC 2 | 12 | 33 | MAC 2 | 12 | 92 |

### Example 6: Dehydration and Anesthesia Influence on the Relationship Between Arterial and Venous Pressure Waveforms

The piezoelectric signal was measured along with the PVP in patients in Examples 1-5 to find if there was any correlation between the two signals. From FIGS. 2A-2B, it is clear that the two waveforms have harmonic peaks at similar frequencies. In FIG. 2C, the harmonic with the highest amplitude is at 2 Hz, which is lower than the frequency, 1.2 Hz, of the highest amplitude in FIG. 2D.

The electrocardiogram (ECG/EKG) was measured along with the PVP in patients in Examples 1-5 to find if there was any correlation between the two waveforms. In FIGS. 3A-3B, the two signals have harmonic peaks at similar frequencies. In FIG. 3C, the harmonic with the highest amplitude is at 1.2 Hz, which is similar to the frequency of the highest amplitude in FIG. 3D.

In addition, data from pediatric patients was collected from 5 sequential patients undergoing surgery for pyloric stenosis. PVP and ECG waveforms were continuously collected from patients before and after the application of the anesthetic, propofol. A porcine dataset was collected on 52 healthy pigs before and after being subjected to slow bleeding. Vital signals including CVP and ECG were recorded.

PVP and ECG waveforms were down sampled to 100 Hz and analyzed with LabChart. Motion artifacts interfering with the peripheral venous pressure waveform were removed with the pre-processing algorithm described above before signal analysis. PVP, CVP and ECG waveforms were sectioned into 2-second snippets, and an FFT was applied. A power spectral density (PSD) was plotted for each snippet and the magnitude of the amplitude of the frequencies F₀, corresponding to the respiration rate and F₁, corresponding to the pulse rate, were calculated in each snippet. A time-domain sample of a CVP and ECG waveform, along with the corresponding power spectral density with F₀ and F₁ labeled and correlation coefficient scatter plot are illustrated in FIGS. 9A-9D.

The Pearson's correlation coefficient was calculated (Eq. 1) between the PVP/CVP and ECG waveforms at the F₀ and F₁ frequencies for each subject.

$${ρ}_{X , Y} = \frac{\sum ( ( X - {μ}_{X} ) ( Y - {μ}_{Y} ) )}{{σ}_{X} {σ}_{Y}} \qquad Eq . 1$$

In the above equation, X is the magnitude of the amplitude at F₀ or F₁ from the PVP/CVP waveform and Y is the magnitude of the amplitude at F₀ or F₁ from the ECG waveform. The corresponding p-values were also recorded and a significance level of 0.05 was used.

FIG. 9A is a two second time series example of porcine CVP waveform before bleeding. FIG. 9B is a simultaneous two second time series example of the porcine ECG waveform before bleeding. FIG. 9C is a power spectral density of the CVP and ECG with respiratory rate, F₀, and pulse rate, F₁, labeled. FIG. 9D is a correlation coefficient plot at F₁. Table 19 shows all Pearson's correlation coefficients and average peak frequency (Hz) at F₀ and F₁.

TABLE 19
Pearson’s correlation coefficients

| | | Highest/ | | Highest/ |
|---|---|---|---|---|
| | Average | Lowest | Average | Lowest |
| | F₀ (Hz) | ρ at F₀ | F₁ (Hz) | ρ at F₁ |
| Animal—Before | 0.21 Hz | 0.95/0.53 | 1.51 Hz | 0.93/0.53 |
| Bleeding | | | | |
| Animal—After | 0.21 Hz | 0.94/0.54 | 1.47 Hz | 0.90/0.52 |
| Bleeding | | | | |
| Human—Before | 0.24 Hz, | 0.39/0.35 | 2.23 Hz | 0.96/0.13 |
| Anesthetic | | | | |
| Human—After | 0.25 Hz | 0.46* | 2.62 Hz | 0.96/0.57 |
| Anesthetic | | | | |

For humans before anesthetics, the average F₀ was 0.24 Hz and the average F₁ was 2.23 Hz. Only two of the five pediatric patients had a correlation coefficient at F₀ with a respective p-value below 0.05 before anesthetic application, and three had coefficients with p-values lower than 0.05 at F₁. The strongest correlation coefficient at frequency F₀ was 0.39 and the weakest 0.35. The strongest correlation coefficient at frequency F₁ was 0.96 and the weakest 0.13.

For humans after anesthetics, the average F₀ was 0.25 Hz and the average F₁ was 2.62 Hz. Only one of the five pediatric patients had a correlation coefficient at F₀ with a respective p-value below 0.05 after anesthetic application, and all five had coefficients with p-values lower than 0.05 at F₁. The coefficient at F₀ was 0.46. The strongest coefficient at F₁ was 0.96 and the weakest 0.57.

For animals before bleeding, the average F₀ was 0.21 Hz and the average F₁ was 1.51 Hz. Out of the fifty-two pigs before bleeding, 22 had correlation coefficients with a p-value below 0.05 at frequency F₁. The strongest coefficient was 0.93 and the weakest 0.53. At F₀ before bleeding, 33 pigs had coefficients with a p-value below 0.05, with the strongest being 0.95 and the weakest being 0.53.

For animals after bleeding, the average F₀ was 0.21 Hz and the average F₁ was 1.47 Hz. After bleeding, 21 of the fifty-two pigs had correlation coefficients with a p-value below 0.05 at frequency F₁. The strongest coefficient was 0.90 and the weakest 0.52. At frequency F₀, 33 pigs had coefficients with a p-value below 0.05, with the strongest being 0.94 and the weakest being 0.54.

This shows that arterial pulse pressure has a strong relationship with PVP waveforms even under the influence of strong pharmacological agents, and CVP even after large blood loss. The correlation coefficients found at F₁ using the PVP waveforms are slightly stronger than those from the CVP waveforms, which is most likely due to the difference in sampling rates between the two datasets. The pediatric dataset had a lower sampling rate of 100 Hz, resulting in an improved quality power spectral density curve for analysis.

Overall, the statistically significant correlation coefficient at F₁ is strongest in the pediatric dataset after anesthetic, which may be because of the dilation of the veins which increases proximity to nearby arteries. The strongest correlation coefficient at F₀ was present in the porcine dataset before bleeding, thus before the vessel diameters decreased due to dehydration.

In the human pediatric dataset, larger variability in the correlation coefficients at F₁ before and after the anesthetic was observed, and the coefficients at F₀ were weak in both situations. The strongest coefficient at F₁ in the pediatric dataset before anesthetic is comparable to the coefficient found after anesthetic application. Surprisingly, the correlation coefficients at F₀ and F₁ are comparable before bleeding and after bleeding in the porcine dataset, but this result does not describe before and after anesthetic, as the pigs were sedated through both stages.

Arterial pressure changes on PVP during the use of an inhaled anesthetic may be analyzed and to look at how specifically the magnitude of the amplitude at F₀ and F₁ is changing before and after anesthetic, as well as before and after mild to severe blood loss.

Having described several embodiments, it will be recognized by those skilled in the art that various modifications, alternative constructions, and equivalents may be used without departing from the spirit of the disclosure. Additionally, a number of well-known processes and elements have not been described in order to avoid unnecessarily obscuring the present disclosure. Accordingly, the above description should not be taken as limiting the scope of the disclosure.

Those skilled in the art will appreciate that the presently disclosed embodiments teach by way of example and not by limitation. Therefore, the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

1. A method of predicting a hemodynamic state of a patient being administered an anesthetic, the method comprising:
receiving a peripheral venous pressure (PVP) waveform from the patient;
cleaning the PVP waveform;
transforming the PVP waveform into the frequency domain; and automatically predicting a hemodynamic state of the patient.

2. The method of claim 1, wherein the hemodynamic state is automatically predicted using a k-nearest neighbor (k-NN), neural network, random forest, SVM, naïve Bayes, and/or K-Means model.

3. The method of claim 1, further comprising acquiring the PVP waveform using a peripheral intravenous catheter linked to a pressure transducer.

4. The method of claim 1, further comprising measuring the patient's electrocardiography (ECG) and determining ECG and PVP waveform coefficients at the heart rate and respiratory rate frequencies.

5. The method of claim 1, wherein cleaning the PVP waveform comprises:
sectioning the PVP waveform at a pre-selected length of time to create one or more segments;
calculating a remainder of the PVP waveform divided by the pre-selected length of time;
removing any last points of the PVP waveform that are equal to the PVP waveform remainder;
calculating the mean and the standard deviation for each segment; and
removing a segment if there is at least one point outside a set number of standard deviations selected by the user.

6. The method of claim 1, wherein the hemodynamic state is a hypervolemic state, an euvolemic state, or a hypovolemic state.

7. The method of claim 1, wherein the anesthetic is an infused anesthetic, and wherein the infused anesthetic is:
an infused GABA agonist selected from propofol, etomidate, and benzodiazepines;
an infused narcotic selected from fentanyl, remifentanil, sufentanyl, morphine, and hydromorphone;
an infused barbiturate selected from phenobarbital, pentobarbital, and methohexital;
an infused NMDA antagonist selected from ketamine and esketamine;
an infused alpha agonist such as precedex; or
an infused neuraxial anesthetic selected from lidocaine, bupivacaine, ropivacaine, tetracaine, chloroprocaine, clonidine, fentanyl, hydromorphone, morphine, epinephrine, sodium bicarbonate, and glucocorticoids.

8.-13. (canceled)

14. The method of claim 1, wherein the patient is a pediatric patient.

15. A method of predicting an anesthetic depth of a patient being administered an anesthetic, the method comprising:
receiving a peripheral venous pressure (PVP) waveform from the patient;
cleaning the PVP waveform;
transforming the PVP waveform into the frequency domain; and
automatically predicting the anesthetic depth of the patient.

16. The method of claim 15, wherein the anesthetic depth is automatically predicted using a k-nearest neighbor (k-NN), neural network, random forest, SVM, naïve Bayes, and/or K-means model.

17. The method of claim 15, further comprising acquiring the PVP waveform using a peripheral intravenous catheter linked to a pressure transducer.

18. The method of claim 15, further comprising measuring the patient's ECG and determining ECG and PVP waveform coefficients at the heart rate and respiratory rate frequencies.

19. The method of claim 15, wherein cleaning the PVP waveform comprises:
sectioning the PVP waveform at a pre-selected length of time to create one or more segments;
calculating a remainder of the PVP waveform divided by the pre-selected length of time;
removing any last points of the PVP waveform that are equal to the PVP waveform remainder;
calculating the mean and the standard deviation for each segment; and
removing a segment if there is at least one point outside a set number of standard deviations selected by the user.

20. The method of claim 15, wherein the anesthetic depth is a minimum alveolar concentration (MAC) dosage.

21. The method of claim 15, wherein the anesthetic is an inhaled anesthetic.

22. The method of claim 21, wherein the inhaled anesthetic is selected from isoflurane, sevoflourane, desflurane, halothane, and nitrous oxide.

23. The method of claim 15, wherein the patient is a pediatric patient.

24. The method of claim 15, further comprising preventing overdosage or underdosage of anesthesia during a medical operation using the predicted anesthetic depth in the patient.

25. The method of claim 24, further comprising providing a minimum and/or maximum anesthetic depth; and adjusting the anesthetic administered to the patient to maintain the predicted anesthetic depth within the minimum and maximum values.

26.-33. (canceled)
